(19) United States
(12) Patent Application Publication
(10) Pub. No.: US 2024/0386558 A1
(43) Pub. Date: Nov. 21, 2024

(54) SYSTEMS AND METHODS FOR BLOOD VESSEL IMAGE PROCESSING

(71) Applicant: SHANGHAI UNITED IMAGING HEALTHCARE CO., LTD., Shanghai (CN)

(72) Inventor: Jian GUO, Shanghai (CN)

(73) Assignee: SHANGHAI UNITED IMAGING HEALTHCARE CO., LTD., Shanghai (CN)

(21) Appl. No.: 18/789,650

(22) Filed: Jul. 30, 2024

Related U.S. Application Data
(63) Continuation of application No. PCT/CN2023/100201, filed on Jun. 14, 2023.

(30) Foreign Application Priority Data
Jun. 14, 2022 (CN) 202210669343.1

(51) Int. Cl. G06T 7/00; G06N 3/044; G16H 30/20

(52) U.S. Cl. CPC G06T 7/0012; G06N 3/044; G16H 30/20; G06T 2207/20081; G06T 2207/30101

(57) ABSTRACT

Systems and methods for image processing are provided. The systems may obtain a blood vessel image of a target subject. The systems may generate, based on the blood vessel image, a point cloud including a plurality of data points representing a plurality of blood vessel points of the target subject. Each of the plurality of data points may include values of one or more reference features of the corresponding blood vessel point. The systems may further for each of the plurality of blood vessel points, determine values of one or more target features of the blood vessel point based on the point cloud using a determination model. The determination model may be a trained deep learning model.

### CROSS-REFERENCE TO RELATED APPLICATIONS

This application is a continuation of International Application No. PCT/CN2023/100201, filed on Jun. 14, 2023, which claims priority to Chinese Patent Application No. 202210669343.1 filed on Jun. 14, 2022, the entire contents of which are hereby incorporated by reference.

### TECHNICAL FIELD

The present disclosure generally relates to image processing, and more particularly, relates to systems and methods for determining values of target features of blood vessel points by processing blood vessel images.

### BACKGROUND

Lesions, for example, different types of plaques or different degrees of stenosis, often occur in blood vessels (e.g., coronary arteries, carotid blood vessels, lower extremity blood vessels, etc.). Values of target features (also referred to as blood flow characteristics) of blood vessel points can reflect the condition of the lesions. Generally, the values of the target features of the blood vessel points can be assessed by artificially observing blood vessel images, which are susceptible to human error or subjectivity. Therefore, it is desirable to provide more accurate systems and methods for determining the values of the target features of the blood vessel points.

### SUMMARY

A further aspect of the present disclosure relates to a method for image processing. The method is implemented on a computing device including at least one processor and at least one storage device. The method includes obtaining a blood vessel image of a target subject; generating, based on the blood vessel image, a point cloud including a plurality of data points representing a plurality of blood vessel points of the target subject, each of the plurality of data points including values of one or more reference features of the corresponding blood vessel point; and for each of the plurality of blood vessel points, determining values of one or more target features of the blood vessel point based on the point cloud using a determination model, wherein the determination model is a trained deep learning model.

In some embodiments, the determination model is obtained by obtaining a plurality of training samples, wherein each of the plurality of training samples includes a sample point cloud representing sample blood vessel points of a sample blood vessel and ground truth values of the one or more target features of the sample blood vessel points, the sample blood vessel corresponding to at least one of the plurality of training samples is a virtual blood vessel; and generating the determination model by training a preliminary deep learning model based on the plurality of training samples.

In some embodiments, the sample point cloud of a virtual blood vessel is determined using a trained generator based on one or more characteristic values of the virtual blood vessel.

In some embodiments, the trained generator is obtained by training, based on a plurality of second training samples, a generative adversarial network (GAN) including a generator and a discriminator, each of the plurality of second training samples including a sample characteristic value of a sample real blood vessel and a sample point cloud representing the sample real blood vessel.

In some embodiments, the sample point cloud of a virtual blood vessel is determined by determining a virtual center line of the virtual blood vessel; for each point of the virtual center line, determining a blood vessel section centered on the point of the virtual center line based on a constraint condition; generating the virtual blood vessel based on blood vessel sections corresponding to points of the virtual center line; and determining the sample point cloud based on the virtual blood vessel.

In some embodiments, the determination model includes a PointNet, a recurrent neural network (RNN), and a determination network. The PointNet is configured to determine local features and global features of the plurality of blood vessel points based on the values of the reference features of the plurality of blood vessel points. The RNN is configured to generate an output by processing the local features of the plurality of blood vessel points. The determination network is configured to generate, for each of the plurality of blood vessel points, the values of the one or more target features of the blood vessel point based on the local features, the global features, and the output of the RNN.

In some embodiments, the determination model includes a point encoder and a point decoder. The point encoder is configured to determine encoded first features of the plurality of blood vessel points and encoded second features of a plurality of blood vessel slices based on first features of the plurality of blood vessel points and second features of the plurality of blood vessel slices. The first features include the values of the reference features of the plurality of blood vessel points. The second features of each blood vessel slice include the values of the reference features of blood vessel points in the blood vessel slice. The point decoder is configured to determine, for each of the plurality of blood vessel points, the values of the one or more target features of the blood vessel point based on a combination of the encoded first features and the encoded second features.

In some embodiments, the determination model further includes a sequence encoder configured to generate central features relating to central points of the plurality of blood vessel slices based on the second features and the encoded second features. The point decoder is further configured to determine the values of the one or more target features of each of the plurality of blood vessel points based on the combination of the encoded first features and the encoded second features, and an up-sampling result of the central features.

In some embodiments, each of a plurality of training samples is configured to train the determination model includes ground truth values of the one or more target features of central points of a sample blood vessel. During the training of the determination model, a preliminary sequence encoder in a preliminary deep learning model is configured to determine predicted values of the one or more target features of the central points of the sample blood vessel in each iteration.

In some embodiments, a loss function used for training the determination model includes a point loss and a sequence loss. The point loss is related to ground truth values of the one or more target features of sample blood vessel points of the sample blood vessel. The sequence loss is related to the ground truth values of the one or more target features of the central points of the sample blood vessel.

In some embodiments, for each of the plurality of blood vessel points, the determining the values of the one or more target features of the blood vessel point based on the point cloud using the determination model includes for each of one or more reference features of the blood vessel point, determining a weight of the reference feature based on a position of the blood vessel point in a blood vessel corresponding to the blood vessel point; and determining the values of the one or more target features of the blood vessel point based on values and weights of reference features of the plurality of blood vessel points using the determination model.

In some embodiments, for each of the plurality of blood vessel points, the determining the values of the one or more target features of the blood vessel point based on the point cloud using the determination model includes dividing one or more reference features of the blood vessel point into a plurality of reference feature sets; for each of the plurality of reference feature sets, determining a weight of the reference feature set based on a position, in a blood vessel corresponding to the blood vessel point, of the blood vessel point; determining a candidate value set of the one or more target features of the blood vessel point based on values of reference features in the reference feature set using the determination model; and determining the values of the one or more target features of the blood vessel point based on candidate value sets and weights corresponding to the plurality of reference feature sets.

Another aspect of the present disclosure relates to a system for image processing. The system includes at least one storage device including a set of instructions and at least one processor in communication with the at least one storage device. When executing the set of instructions, the at least one processor is directed to cause the system to implement operations. The operations include obtaining a blood vessel image of a target subject; generating, based on the blood vessel image, a point cloud including a plurality of data points representing a plurality of blood vessel points of the target subject, each of the plurality of data points including values of one or more reference features of the corresponding blood vessel point; and for each of the plurality of blood vessel points, determining values of one or more target features of the blood vessel point based on the point cloud using a determination model, wherein the determination model is a trained deep learning model.

A further aspect of the present disclosure relates to a system for image processing. The system includes an obtaining module, a generation module, and a determination module. The obtaining module is configured to obtain a blood vessel image of a target subject. The generation module is configured to generate, based on the blood vessel image, a point cloud including a plurality of data points representing a plurality of blood vessel points of the target subject, each of the plurality of data points including values of one or more reference features of the corresponding blood vessel point. The determination module is configured to for each of the plurality of blood vessel points, determine values of one or more target features of the blood vessel point based on the point cloud using a determination model, wherein the determination model is a trained deep learning model.

A still further aspect of the present disclosure relates to a non-transitory computer readable medium including executable instructions. When the executable instructions are executed by at least one processor, the executable instructions direct the at least one processor to perform a method. The method includes obtaining a blood vessel image of a target subject; generating, based on the blood vessel image, a point cloud including a plurality of data points representing a plurality of blood vessel points of the target subject, each of the plurality of data points including values of one or more reference features of the corresponding blood vessel point; and for each of the plurality of blood vessel points, determining values of one or more target features of the blood vessel point based on the point cloud using a determination model, wherein the determination model is a trained deep learning model.

Additional features may be set forth in part in the description which follows, and in part may become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIGS. 2A and 2B are block diagrams illustrating exemplary processing devices according to some embodiments of the present disclosure;

FIG. 3 is a flowchart illustrating an exemplary process for determining values of target features of blood vessel points according to some embodiments of the present disclosure;

FIG. 4 is a schematic diagram illustrating an exemplary blood vessel image according to some embodiments of the present disclosure;

FIG. 5 is a schematic diagram illustrating exemplary local point set features of a blood vessel point according to some embodiments of the present disclosure;

FIG. 6A is a schematic diagram illustrating an exemplary process for determining value of target value(s) of a blood vessel point according to some embodiments of the present disclosure;

FIG. 6B is a schematic diagram illustrating exemplary reference feature sets according to some embodiments of the present disclosure;

FIG. 7 is a flowchart illustrating an exemplary process for determining a determination model according to some embodiments of the present disclosure;

FIG. 8 is a schematic diagram illustrating an exemplary process for determining a first sample point cloud according to some embodiments of the present disclosure;

FIG. 9 is a schematic diagram illustrating an exemplary process for determining a trained generator according to some embodiments of the present disclosure;

FIG. 10 is a schematic diagram illustrating an exemplary process for determining a first sample point cloud according to some embodiments of the present disclosure;

FIG. 11 is a schematic diagram illustrating an exemplary determination model according to some embodiments of the present disclosure; and

FIG. 12 is a schematic diagram illustrating exemplary blood vessel slices according to some embodiments of the present disclosure.

### DETAILED DESCRIPTION

In the following detailed description, numerous specific details may be set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments may be readily apparent to those skilled in the art, and the general principles defined herein may be applied to other embodiments and applications without departing from the spirit and scope of the present disclosure. Thus, the present disclosure may be not limited to the embodiments shown, but to be accorded the widest scope consistent with the claims.

The terminology used herein may be for the purpose of describing particular example embodiments only and may be not intended to be limiting. As used herein, the singular forms “a,” “an,” and “the” may be intended to include the plural forms as well, unless the context clearly indicates otherwise. It may be understood that the terms “system,” “unit,” “module,” and/or “block” used herein are one method to distinguish different components, elements, parts, sections or assemblies of different levels in ascending order. However, the terms may be displaced by another expression if they achieve the same purpose.

The modules (or units, blocks, units) described in the present disclosure may be implemented as software and/or hardware modules and may be stored in any type of non-transitory computer-readable medium or other storage devices. In some embodiments, a software module may be compiled and linked into an executable program. It may be appreciated that software modules may be callable from other modules or from themselves, and/or may be invoked in response to detected events or interrupts. Software modules configured for execution on computing devices may be provided on a computer readable medium or as a digital download (and can be originally stored in a compressed or installable format that requires installation, decompression, or decryption prior to execution). Such software code may be stored, partially or fully, on a memory device of the executing computing device, for execution by the computing device. Software instructions may be embedded in a firmware, such as an EPROM. It may be further appreciated that hardware modules (e.g., circuits) may be included in connected or coupled logic units, such as gates and flip-flops, and/or may be included in programmable units, such as programmable gate arrays or processors. The modules or computing device functionality described herein may be preferably implemented as hardware modules, but may be software modules as well. In general, the modules described herein refer to logical modules that may be combined with other modules or divided into units despite their physical organization or storage.

Certain terminology has been used to describe embodiments of the present disclosure. For example, the terms “one embodiment,” “an embodiment,” and/or “some embodiments” may mean that a particular feature, structure or characteristic described in connection with the embodiment is in at least one embodiment of the present disclosure. Therefore, it is emphasized and should be appreciated that two or more references to “an embodiment” or “one embodiment” or “an alternative embodiment” in various portions of this specification may not necessarily all referring to the same embodiment. Furthermore, the particular features, structures or characteristics may be combined as suitable in one or more embodiments of the present disclosure.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings may be for the purpose of illustration and description only and may be not intended to limit the scope of the present disclosure.

The flowcharts used in the present disclosure may illustrate operations that systems implement according to some embodiments of the present disclosure. It is to be expressly understood, the operations of the flowcharts may be implemented not in order. Conversely, the operations may be implemented in inverted order, or simultaneously. Moreover, one or more other operations may be added to the flowcharts. One or more operations may be removed from the flowcharts.

The present disclosure provides systems and methods for blood vessel image processing. The systems may obtain a blood vessel image of a target subject (e.g., a patient). The systems may generate a point cloud based on the blood vessel image. The point cloud may include a plurality of data points representing a plurality of blood vessel points of the target subject. Each of the plurality of data points including values of one or more reference features of the corresponding blood vessel point. For each of the plurality of blood vessel points, the systems may determine values of one or more target features of the blood vessel point based on the point cloud using a determination model. The determination model may be a trained deep learning model.

According to the embodiments of the present disclosure, the values of the target features of each blood vessel point may be automatically determined based on the point cloud including values of reference features of all blood vessel points of the target subject. Compared with determining the values of the target features by artificially observing the blood vessel image, the methods disclosed herein are more reliable and robust, insusceptible to human error or subjectivity, and/or fully automated.

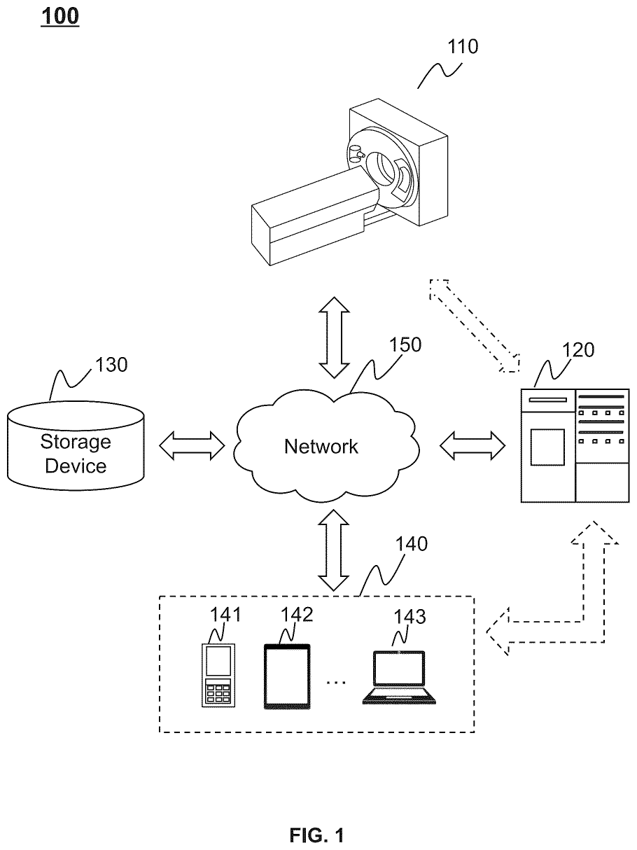

FIG. 1 is a schematic diagram illustrating an exemplary medical system 100 according to some embodiments of the present disclosure. As shown in FIG. 1, the medical system 100 may include an imaging device 110, a processing device 120, a storage device 130, one or more terminals 140, and a network 150. In some embodiments, the imaging device 110, the processing device 120, the storage device 130, and/or the terminal(s) 140 may be connected to and/or communicate with each other via a wireless connection, a wired connection, or a combination thereof.

The imaging device 110 may be configured to scan a target subject (or a part of the subject) to acquire medical image data associated with the target subject. The medial image data relating to the target subject may be used for generating an anatomical image (e.g., a CT image, an MRI image) (e.g., a blood vessel image) of the target subject. The anatomical image may illustrate an internal structure (e.g., blood vessels) of the target subject.

In some embodiments, the imaging device 110 may include a single-modality scanner and/or multi-modality scanner. The single modality scanner may include, for example, an X-ray scanner, a CT scanner, a magnetic resonance imaging (MRI) scanner, an ultrasonography scanner, a positron emission tomography (PET) scanner, a Digital Radiography (DR) scanner, or the like, or any combination thereof. The multi-modality scanner may include, for example, an X-ray imaging-magnetic resonance imaging (X-ray-MRI) scanner, a positron emission tomography-X-ray imaging (PET-X-ray) scanner, a single-photon emission computed tomography-magnetic resonance imaging (SPECT-MRI) scanner, a positron emission tomography-computed tomography (PET-CT) scanner, etc. It should be noted that the imaging device 110 described below is merely provided for illustration purposes, and not intended to limit the scope of the present disclosure.

In some embodiments, the processing device 120 may be a single server or a server group. The server group may be centralized or distributed. The processing device 120 may process data and/or information obtained from the imaging device 110, the storage device 130, and/or the terminal(s) 140. For example, the processing device 120 may determine values of one or more target features of blood vessel points of a target subject. As another example, the processing device 120 may generate one or more deep learning models (e.g., a determination model) used for determining the values of the target feature(s).

In some embodiments, the processing device 120 may be local or remote from the medical system 100. In some embodiments, the processing device 120 may be implemented on a cloud platform. In some embodiments, the processing device 120 or a portion of the processing device 120 may be integrated into the imaging device 110 and/or the terminal(s) 140. It should be noted that the processing device 120 in the present disclosure may include one or multiple processors. Thus operations and/or method steps that are performed by one processor may also be jointly or separately performed by the multiple processors.

The storage device 130 may store data (e.g., the blood vessel image, the point cloud, the values of the one or more target features of each blood vessel point, the determination model, etc.), instructions, and/or any other information. In some embodiments, the storage device 130 may store data obtained from the imaging device 110, the processing device 120, and/or the terminal(s) 140. In some embodiments, the storage device 130 may store data and/or instructions that the processing device 120 may execute or use to perform exemplary methods described in the present disclosure. In some embodiments, the storage device 130 may include a mass storage device, a removable storage device, a volatile read-and-write memory, a read-only memory (ROM), or the like, or a combination thereof. In some embodiments, the storage device 130 may be implemented on a cloud platform. In some embodiments, the storage device 130 may be part of the imaging device 110, the processing device 120, and/or the terminal(s) 140.

The terminal(s) 140 may be configured to enable a user interaction between a user and the medical system 100. In some embodiments, the terminal(s) 140 may be connected to and/or communicate with the imaging device 110, the processing device 120, and/or the storage device 130. In some embodiments, the terminal(s) 140 may include a mobile device 141, a tablet computer 142, a laptop computer 143, or the like, or a combination thereof. In some embodiments, the terminal(s) 140 may be part of the processing device 120 and/or the Imaging device 110.

The network 150 may include any suitable network that can facilitate the exchange of information and/or data for the medical system 100. In some embodiments, one or more components of the medical system 100 (e.g., the imaging device 110, the processing device 120, the storage device 130, the terminal(s) 140, etc.) may communicate information and/or data with one or more other components of the medical system 100 via the network 150.

It should be noted that the above description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. In some embodiments, the medical system 100 may include one or more additional components and/or one or more components described above may be omitted.

Additionally or alternatively, two or more components of the medical system 100 may be integrated into a single component. For example, the processing device 120 may be integrated into the imaging device 110. As another example, a component of the medical system 100 may be replaced by another component that can implement the functions of the component. However, those variations and modifications do not depart from the scope of the present disclosure.

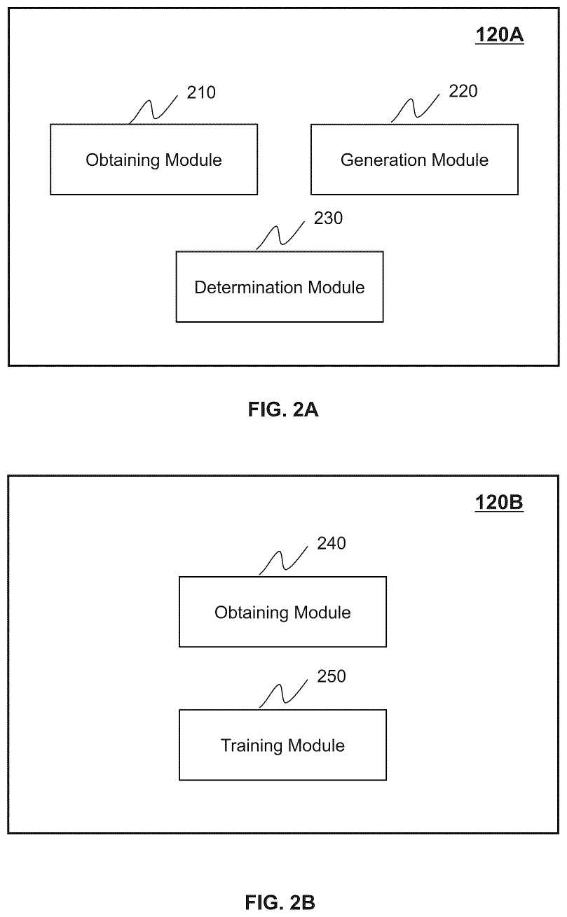

FIGS. 2A and 2B are block diagrams illustrating exemplary processing devices 120A and 120B according to some embodiments of the present disclosure. The processing devices 120A and 120B may be exemplary processing devices 120 as described in connection with FIG. 1. In some embodiments, the processing device 120A may be configured to apply a determination model for determining values of one or more target features of blood vessel points. The processing device 120B may be configured to obtain a plurality of training samples and/or determine one or more models (e.g., the determination model) using the training samples. In some embodiments, the processing devices 120A and 120B may be respectively implemented on a processing unit (e.g., a processor of the processing device 120). Alternatively, the processing devices 120A and 120B may be implemented on a same computing unit.

As shown in FIG. 2A, the processing device 120A may include an obtaining module 210, a generation module 220, and a determination module 230.

The obtaining module 210 may be configured to obtain a blood vessel image of a target subject. More descriptions regarding the obtaining of the blood vessel image may be found elsewhere in the present disclosure (e.g., operation 310 and the description thereof).

The generation module 220 may be configured to generate a point cloud based on the blood vessel image. More descriptions regarding the generation of the point cloud may be found elsewhere in the present disclosure (e.g., operation 320 and the description thereof).

The determination module 230 may be configured to determine values of one or more target features of the blood vessel point based on the point cloud using a determination model. More descriptions regarding the determination of the values of the one or more target features of the blood vessel point may be found elsewhere in the present disclosure (e.g., operation 330 and the description thereof).

As shown in FIG. 2B, the processing device 120B may include an obtaining module 240 and a training module 250.

The obtaining module 240 may be configured to obtain a plurality of training samples. More descriptions regarding the obtaining of the plurality of training samples may be found elsewhere in the present disclosure (e.g., operation 710 and the description thereof).

The training module 250 may be configured to generate the determination model by training a preliminary deep learning model based on the plurality of training samples. More descriptions regarding the generation of the determination model may be found elsewhere in the present disclosure (e.g., operation 720 and the description thereof).

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the processing device 120A and/or the processing device 120B may share two or more of the modules, and any one of the modules may be divided into two or more units. For instance, the processing devices 120A and 120B may share a same obtaining module; that is, the obtaining module 210 and the obtaining module 240 are a same module. In some embodiments, the processing device 120A and/or the processing device 120B may include one or more additional modules, such as a storage module (not shown) for storing data. In some embodiments, the processing device 120A and the processing device 120B may be integrated into one processing device 120.

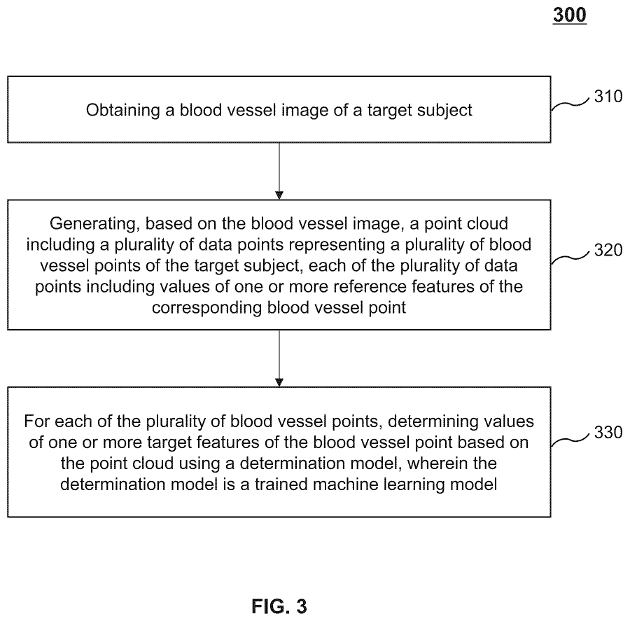

FIG. 3 is a flowchart illustrating an exemplary process for determining values of target features of blood vessel points according to some embodiments of the present disclosure. In some embodiments, process 300 may be executed by the medical system 100. For example, the process 300 may be implemented as a set of instructions (e.g., an application) stored in a storage device (e.g., the storage device 130). In some embodiments, the processing device 120A (e.g., one or more modules illustrated in FIG. 2A) may execute the set of instructions and may accordingly be directed to perform the process 300.

In 310, the processing device 120A (e.g., the obtaining module 210) may obtain a blood vessel image of a target subject.

In some embodiments, the target subject may include a human being (e.g., a patient), an animal, or a specific portion, organ, and/or tissue thereof. Merely by way of example, the target subject may include head, chest, abdomen, heart, liver, upper limbs, lower limbs, or the like, or any combination thereof. In the present disclosure, the term “object” or “subject” are used interchangeably in the present disclosure.

The blood vessel image may refer to an image including blood vessels of the target subject. Merely by way of example, the blood vessels may be located in various parts of the target subject, for example, head, neck, abdomen, lower extremities, etc. Merely by way of example, the blood vessels include vertebral artery, basilar artery, internal carotid artery, coronary arteries, abdominal aorta, renal artery, hepatic portal vein, deep veins, superficial veins, communicating veins of the lower extremities, and muscle veins of the lower extremities, etc. In some embodiments, a format of the blood vessel image may include, for example, a joint photographic experts group (JPEG) format, a tag image file format (TIFF), a graphics interchange format (GIF), a digital imaging and communications in medical (DICOM) format, etc. In some embodiments, the blood vessel image may include a two-dimensional (2D) image, a three-dimensional (3D) image, a four-dimensional (4D) image, etc.

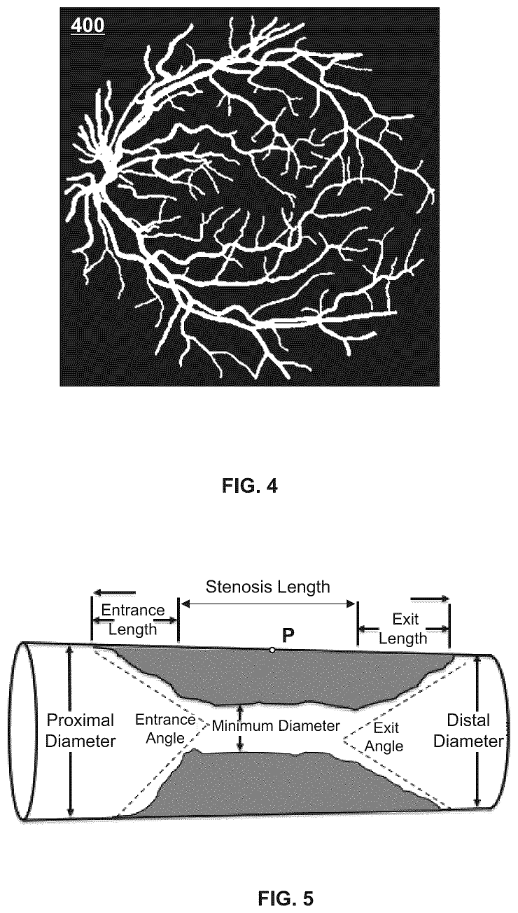

FIG. 4 is a schematic diagram illustrating an exemplary blood vessel image according to some embodiments of the present disclosure. As shown in FIG. 4, a blood vessel image 400 may be a binary mask image of blood vessels. In the blood vessel image 400, the blood vessels and the background are represented by different colors. For example, the blood vessels are represented in white, and the background is represented in black.

In some embodiments, the processing device 120A may obtain the blood vessel image of the target subject by directing or causing the imaging device 110 to perform a scan on the target subject. For example, the processing device 120A may direct or cause the imaging device 110 to perform a scan on the target subject to obtain an initial image (e.g., an MRI image, a CT image, a PET image, or the like, or any combination thereof) of the target subject. Further, the processing device 120A may generate the blood vessel image of the target subject by processing the initial image. For example, the processing device 120A may generate the blood vessel image of the target subject by segmenting the blood vessels from the initial image. Merely by way of example, the processing device 120A may segment the initial image by inputting the initial image into a segmentation network, for example, a convolutional neural network, a recurrent neural network, etc. In some embodiments, the blood vessel image of the target subject may be previously obtained and stored in a storage device (e.g., the storage device 130) disclosed elsewhere in the present disclosure and/or an external storage device. The processing device 120 may obtain the blood vessel image of the target subject from the storage device and/or the external storage device via a network (e.g., the network 150).

In 320, the processing device 120A (e.g., the generation module 220) may generate a point cloud based on the blood vessel image.

In some embodiments, the point cloud may include a plurality of data points representing a plurality of blood vessel points of the target subject. Each of the plurality of data points may include values of one or more reference features of the corresponding blood vessel point. The reference feature(s) may include any feature that can provide reference information for determining values of target feature(s) of the blood vessel points. In some embodiments, the one or more reference features may include at least one of a spatial feature, a structure feature, a blood flow feature, or a local point set feature.

The spatial feature of a blood vessel point may refer to a feature related to a spatial position of the blood vessel point. Merely by way of example, the spatial feature may include a spatial coordinate, a normal spatial feature, etc. The normal spatial feature may refer to a normal direction from a centerline of a blood vessel where the blood vessel point is located to the blood vessel point.

The structure feature of a blood vessel point may refer to a feature related to a structure of a portion of the blood vessels where the blood vessel point is located. Merely by way of example, the structure feature may include at least one of a diameter, a cross-sectional area of the vessel, a stenosis rate, or a curvature, of the portion where the blood vessel point is located in the blood vessels. The stenosis rate may refer to a degree of depression of the portion where the blood vessel point is located in the blood vessels. The curvature may refer to a degree of curvature of the portion where the blood vessel point is located in the blood vessels.

The blood flow feature of a blood vessel point may refer to a feature related to blood flow at the blood vessel point. Merely by way of example, the blood flow feature may include at least one of a blood pressure feature, a transport feature, or a mechanics feature at the blood vessel point. The blood pressure feature may refer to a pressure of the blood flow acting on a vessel wall at the blood vessel point. The transport feature may refer to a feature related to the blood flowing at the blood vessel point, for example, a blood flow velocity (e.g., an average blood flow velocity, a maximum blood flow velocity, etc.), a blood viscosity, etc. The mechanics feature may refer to a feature related to a force borne by the blood vessel point, for example, a shear stress.

The local point set feature of a blood vessel point may refer to a feature related to a blood vessel segment where the blood vessel point is located. FIG. 5 is a schematic diagram illustrating exemplary local point set features of a blood vessel point according to some embodiments of the present disclosure. Merely by way of example, as shown in FIG. 5, the local point set feature of a blood vessel point P may include a stenosis length, a proximal diameter, a distal diameter, an entrance angle, an entrance length, an exit angle, an exit length, a cross-sectional area (not shown) of a narrowest part, a maximum diameter (not shown), a minimum diameter, and a stenosis rate (not shown) of a vessel segment where the blood vessel point P is located, etc. The proximal diameter may refer to a diameter of an end (also referred to as a proximal end) of the vessel segment that is closer to the heart. The distal diameter may refer to a diameter of an end (also referred to as a distal end) of the vessel segment that is farther from the heart. The entrance angle may refer to an angle formed by a narrow part of the vessel segment near the proximal end. The exit angle may refer to an angle formed by a narrow part of the vessel segment near the distal end. The entrance length may refer to a length of the narrow part of the vessel segment near the proximal end. The exit length may refer to a length of the narrow part of the vessel segment near the distal end. The stenosis rate may refer to a maximum value among the stenosis rates of blood vessel points constituting the blood vessel segment.

In some embodiments, the value of a reference feature of a blood vessel point may be input by a user (e.g., a doctor, an expert, etc.) manually.

In some embodiments, the processing device 120A may determine the value of a reference feature (e.g., the spatial feature, the structure feature, the blood flow feature, the local point set feature) of a blood vessel point based on the blood vessel image using a feature generation model corresponding to the reference feature. The feature generation model may be a trained deep learning model. For example, the processing device 120A may determine the blood flow velocity of each blood vessel point in the blood vessel image by inputting the blood vessel image into a feature generation model corresponding to the blood flow velocity. The feature generation model may be trained based on training samples with labels. The training samples with labels may include sample blood vessel images in which each blood vessel point is labeled with the value of the reference feature. Based on the training samples with the labels, an initial deep learning model may be iteratively trained to optimize its model parameters, thereby generating the feature generation model.

In some embodiments, the processing device 120A may determine the spatial feature and/or the local point set feature of a blood vessel point based on the blood vessel image. For example, the processing device 120A may determine the spatial coordinate of the blood vessel point based on a position of a pixel corresponding to the blood vessel point in the blood vessel image. As another example, the processing device 120A may establish a three-dimensional model of a blood vessel or a blood vessel segment where the blood vessel point is located based on the blood vessel image, and obtain the spatial coordinate and/or the local point set feature of the blood vessel point based on the three-dimensional model. As yet another example, the processing device 120A may determine the centerline of the blood vessel where the blood vessel point is located based on the blood vessel image, and project the blood vessel point vertically onto the centerline of the blood vessel. Further, the processing device 120A may designate a direction from the projected point to the blood vessel point as the normal spatial feature of the blood vessel point.

In some embodiments, the processing device 120A may determine the structure feature of a blood vessel point based on the spatial feature of the blood vessel point. For example, the processing device 120A may determine a contour of a blood vessel section where the blood vessel point is located based on spatial coordinates of multiple blood vessel points located in a same section. According to the contour of the blood vessel section, the processing device 120A may determine the diameter and/or the cross-sectional area of the portion where the blood vessel point is located in the blood vessels. As another example, the processing device 120A may determine a normal distance between the blood vessel point and the centerline of the blood vessel where the blood vessel point is located along the normal direction corresponding to the blood vessel point. Further, the processing device 120A may determine the stenosis rate and/or the curvature of the portion where the blood vessel point is located in the blood vessel based on the normal distance. The smaller the normal distance, the higher the stenosis rate and/or the curvature.

In 330, for each of the plurality of blood vessel points, the processing device 120A (e.g., the determination module 230) may determine values of one or more target features of the blood vessel point based on the point cloud using a determination model.

The target feature(s) may include any feature of a blood vessel point that is different from the reference feature(s) mentioned above. In some embodiments, the reference feature(s) may include a portion of the spatial feature, the structure feature, the blood flow feature, and the local point set feature, and the one or more target features may include the other portion of the spatial feature, the structure feature, the blood flow feature, and the local point set feature. For example, the reference feature(s) may include the spatial feature, the structure feature, and the local point set feature, and the one or more target features may include the blood flow feature. As another example, the reference feature(s) may include the spatial feature, the structure feature, the local point set feature, and a portion of the blood flow feature, and the one or more target features may include the other portion of the blood flow feature. As yet another example, the reference feature(s) may include a portion of the blood pressure feature, the transport feature, and the mechanics feature, and the one or more target features may include the other portion of the blood pressure feature, the transport feature, and the mechanics feature. Merely by way of example, the blood pressure feature may include the blood pressure feature and the transport feature, and the one or more target features may include the mechanics feature. As another example, the blood pressure feature may include the blood pressure feature, and the one or more target features may include the transport feature and the mechanics feature.

In some embodiments, the one or more target features may include a fractional flow reserve (FFR). The FFR may refer to a ratio of a maximum blood flow through an artery with a stenosis to a maximum blood flow through the artery in the hypothetical absence of the stenosis. For example, FFR may be determined as a ratio of an average pressure (Pd) of a coronary artery at a distal end of the stenosis to an average pressure (Pa) of an aorta at a coronary ostium under a state of maximum myocardial hyperemia. FFR may be used to evaluate coronary artery lesions and the impact of stenosis caused by coronary artery lesions on downstream blood supply.

In some embodiments, the determination model may be a trained deep learning model. For each of the plurality of blood vessel points, the processing device 120A may determine the values of the one or more target features of the blood vessel point by inputting the point cloud (e.g., values of reference features of the plurality of blood vessel points) into the determination model. In some embodiments, the processing device 120A may generate a feature sequence by associating the values of the reference feature(s) of the plurality of blood vessel points based on a blood flow direction of a blood vessel where the plurality of blood vessel points are located, and determine the values of the one or more target features of the blood vessel point by inputting the feature sequence into the determination model. In some embodiments, the processing device 120B may determine the determination model by a training process. For example, the processing device 120B may obtain a plurality of training samples and generate the determination model by training a preliminary deep learning model based on the plurality of training samples. More descriptions regarding the training process may be found elsewhere in the present disclosure (e.g., FIG. 7 and the description thereof).

In some embodiments, the determination model may include a PointNet and a determination network. The PointNet may be configured to determine local features and global features of the plurality of blood vessel points based on the values of the reference features of the plurality of blood vessel points. For example, for each of the plurality of blood vessel points, the PointNet may output local features of the blood vessel point by performing feature extraction and/or transformation on the values of one or more reference features of the blood vessel point, and output global features of the blood vessel point by performing a max pooling on the local features of the blood vessel point. The determination network may be configured to determine, for each of the plurality of blood vessel points, the values of the one or more target features of the blood vessel point based on the local features and the global features.

In some embodiments, the determination model may include the PointNet, a recurrent neural network (RNN), and the determination network. The RNN may be configured to generate an output by processing the local features of the plurality of blood vessel points. For example, the RNN may generate the output by sequencing the local features of the plurality of blood vessel points. The determination network may be configured to generate, for each of the plurality of blood vessel points, the values of the one or more target features of the blood vessel point based on the local features, the global features, and the output of the RNN.

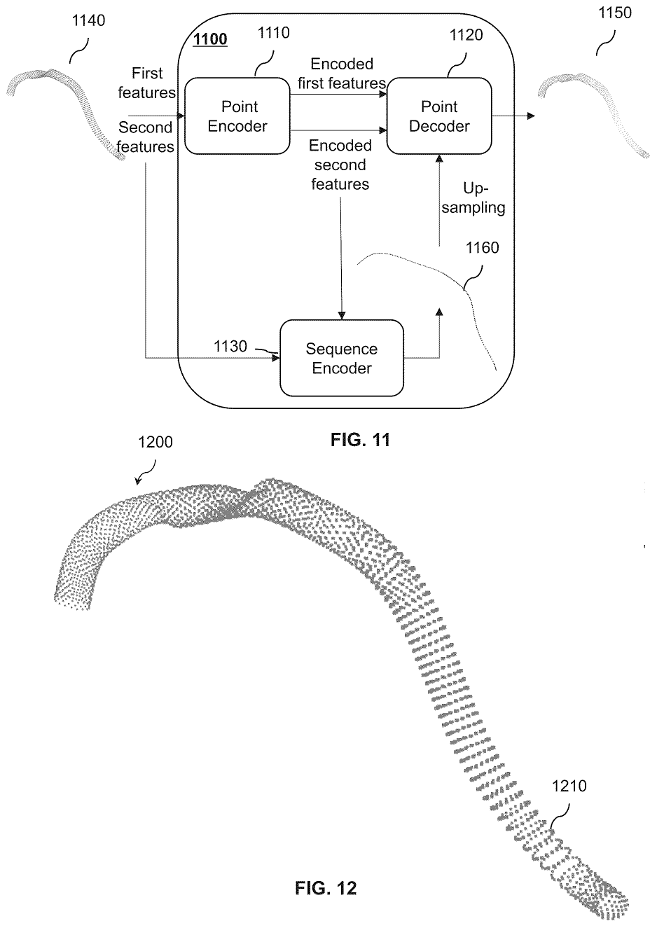

In some embodiments, the determination model may include a point encoder and a point decoder. The point encoder may be configured to determine encoded first features of the plurality of blood vessel points and encoded second features of a plurality of blood vessel slices based on first features of the plurality of blood vessel points and second features of the plurality of blood vessel slices. The point decoder may be configured to determine, for each of the plurality of blood vessel points, the values of the one or more target features of the blood vessel point based on a combination of the encoded first features and the encoded second features. In some embodiments, the determination model may include the point encoder, a sequence encoder, and the point decoder. The sequence encoder may be configured to generate central features relating to central points of the plurality of blood vessel slices based on the second features and the encoded second features. The point decoder may be further configured to determine the values of the one or more target features of each of the plurality of blood vessel points based on combination of the encoded first features and the encoded second features and an up-sampling result of the central features. More descriptions regarding the determination model may be found elsewhere in the present disclosure (e.g., FIG. 11, FIG. 12, and the description thereof).

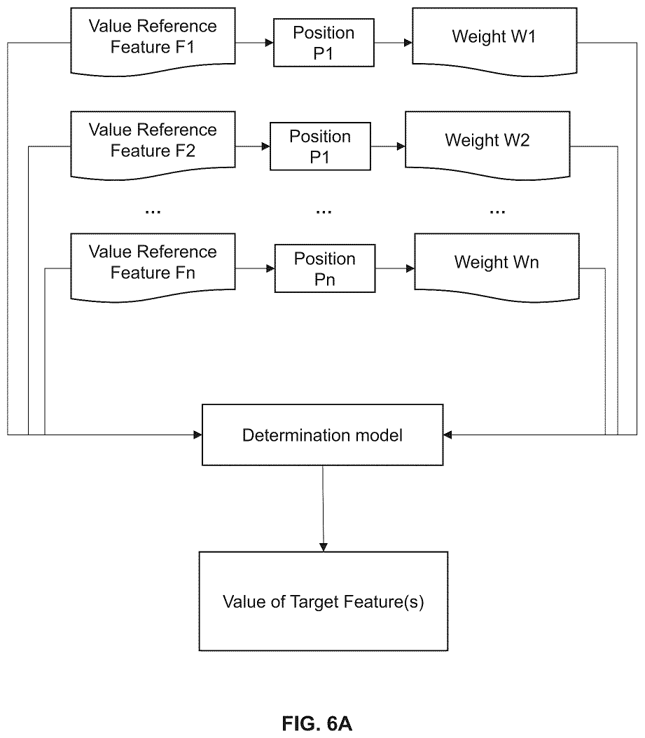

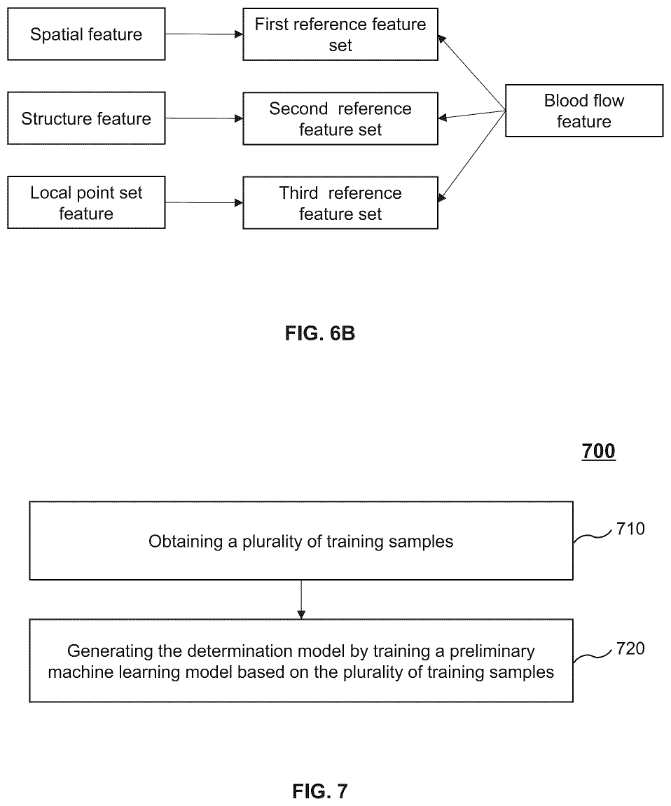

In some embodiments, as shown in FIG. 6A, for each of one or more reference features of the blood vessel point, the processing device 120A may determine a weight of the reference feature based on a position of the blood vessel point in a blood vessel corresponding to the blood vessel point. For example, the closer the blood vessel point is to a starting point/trunk of the blood vessel where the blood vessel point is located, the greater the weight of the spatial feature. As another example, when the blood vessel point is located in a stenosis of the blood vessel where the blood vessel point is located, the weights of the spatial feature and the local point set feature are greater than that of other features in the one or more reference features of the blood vessel point. For blood vessel points in different locations, different reference features have different importance. For example, for a blood vessel point located in a stenosis of a blood vessel, the spatial feature and the local point set feature have a greater impact on FFR than other reference features. Therefore, for the blood vessel point located in the stenosis of the blood vessel, the spatial feature and the local point set feature are assigned with greater weights, which may improve the accuracy of the subsequently determined values of the one or more target features of the blood vessel point.

For example, as shown in FIG. 5, for the blood vessel point P located in a stenosis of a blood vessel, the weight of the spatial feature (e.g., the spatial coordinates) and the local point set feature (e.g., the stenosis length) of the blood vessel point P is greater than other features (e.g., the structure feature, the blood flow feature) of the blood vessel point P. Further, the processing device 120A may determine the values of the one or more target features of the blood vessel point based on values and weights of reference features of the plurality of blood vessel points using the determination model. Merely by way of example, as shown in FIG. 6A, the values of reference features F1-Fn of a blood vessel point and their corresponding weights W1-Wn may be input into the determination model, and the determination model may output the value of the target feature(s) of the blood vessel point. In some embodiments, the values of the reference features and the weights corresponding to multiple blood vessel points may be input into the determination model, and the determination model may determine the values of the target value(s) of the multiple blood vessel points.

In some embodiments, the processing device 120A may divide the one or more reference features of the blood vessel point into a plurality of reference feature sets. For example, the processing device 120A may divide the one or more reference features of the blood vessel point into the plurality of reference feature sets by arbitrarily combining the spatial feature, the structure feature, the blood flow feature, and the local point set feature. Merely by way of example, the processing device 120A may combine any two of the spatial feature, the structure feature, the blood flow feature, and the local point set feature to obtain six reference feature sets. As another example, for the blood vessel points, the blood flow feature is more important than other reference features, accordingly, the processing device 120A may divide the one or more reference features of the blood vessel point into the plurality of reference feature sets by combining the blood flow feature with at least one of the spatial feature, the structure feature, or the local point set feature, thereby improving the accuracy of the subsequently determined values of the one or more target features of the blood vessel points. FIG. 6B is a schematic diagram illustrating exemplary reference feature sets according to some embodiments of the present disclosure. As shown in FIG. 6B, the processing device 120A may combine the blood flow feature with each of the spatial feature, the structure feature, or the local point set feature to obtain a first reference feature set, a second reference feature set, and a third reference feature set.

For each of the plurality of reference feature sets, the processing device 120A may determine a weight of the reference feature set based on a position of the blood vessel point in a blood vessel corresponding to the blood vessel point. For example, the closer the blood vessel point is to the starting point/trunk of the blood vessel where the blood vessel point is located, the greater the weight of the reference feature set including the spatial feature. As another example, when the blood vessel point is located in a stenosis of the blood vessel where the blood vessel point is located, the weight of the reference feature set that includes the spatial feature and/or the local point set feature is greater than other the reference feature sets that do not include the spatial feature and the local point set feature.

For each of the plurality of reference feature sets, the processing device 120A may determine a candidate value set of the one or more target features of the blood vessel point based on values of reference features in the reference feature set using the determination model. In some embodiments, the determination model may include a plurality of sub-models each of which corresponds to a reference feature set. Different reference feature sets may correspond to different sub-models. For each of the plurality of reference feature sets, the processing device 120A may determine the candidate value set of the one or more target features of the blood vessel point based on values of the reference features in the reference feature set using a sub-model corresponding to the reference feature set. Further, the processing device 120A may determine the values of the one or more target features of the blood vessel point based on candidate value sets and weights corresponding to the plurality of reference feature sets. For example, the processing device 120A may determine the values of the one or more target features of the blood vessel point by determining a weighted sum of the candidate value sets corresponding to the plurality of reference feature sets based on the weights corresponding to the plurality of reference feature sets.

In the present disclosure, during the process of determining the values of the one or more target features of the blood vessel point using the determination model, the determination model may mine relationships among the reference features in the point cloud, and those relationships are difficult to be obtained by a traditional manner or an artificial manner of determining the values of the one or more target features of the blood vessel point, thereby improving the accuracy of the determined values of the one or more target features of the blood vessel point.

FIG. 7 is a flowchart illustrating an exemplary process for determining a determination model according to some embodiments of the present disclosure. In some embodiments, the process 700 may be performed to achieve at least part of operation 330 as described in connection with FIG. 3.

In 710, the processing device 120B (e.g., the obtaining module 240) may obtain a plurality of training samples.

In some embodiments, each of the plurality of training samples may include a sample point cloud representing sample blood vessel points of a sample blood vessel and ground truth values of the one or more target features of the sample blood vessel points. The sample blood vessel of a training sample may be a virtual blood vessel or a real blood vessel. As used herein, the real blood vessel may refer to a blood vessel that really exists in a real target subject. The virtual blood vessel may refer to a blood vessel that does not really exist, but is fictitious or simulated based on some means. In some embodiments, the sample blood vessel of at least one training sample may be a virtual blood vessel.

For each training sample, the sample point cloud of the training sample may include values of one or more reference features (e.g., a spatial feature, a structure feature, a blood flow feature, and/or a local point set feature) of each sample blood vessel point of the training sample. When the sample blood vessel corresponding to a training sample is a virtual blood vessel, the sample point cloud of the training sample may be referred to as a first sample point cloud. When the sample blood vessel corresponding to a training sample is a real blood vessel, the sample point cloud corresponding to the training sample may be referred to as a second sample point cloud. The processing device 120B may determine the second sample point cloud based on a blood vessel image (e.g., a historical medical image) of the real blood vessel, for example, in a similar manner as how the point cloud is generated as discussed in FIG. 3. When the count of available second sample point clouds is small or the cost of obtaining second sample point clouds is high, the use of first sample point clouds may increase the number of the training samples of the determination model and reduce the cost of obtaining the training samples, thereby improving the accuracy of the determined determination model.

In some embodiments, the processing device 120B may determine a first sample point cloud of a virtual blood vessel using a trained generator based on one or more characteristic values of the virtual blood vessel. The one or more characteristic values of the virtual blood vessel may relate to one or more parameters of the virtual blood vessel. Merely by way of example, the one or more parameters may include at least one of a length, a diameter, a diameter distribution, a wall thickness, a start position, an end position, a curvature distribution, or lesion data of the virtual blood vessel, a function representing the diameter distribution, or a function representing curvature distribution. The lesion data may include information related to a stenosis in the virtual blood vessel, for example, whether there is a stenosis in the virtual blood vessel, a ratio (e.g., 10%˜90%) of the stenosis to the whole virtual blood vessel, a length of the stenosis, a position of the stenosis, a degree (e.g., a ratio of the diameter of the virtual blood vessel when it includes stenosis to the diameter of the virtual blood vessel when it does not include any stenosis) (e.g., 30%, 50%, 75%) of the stenosis, etc.

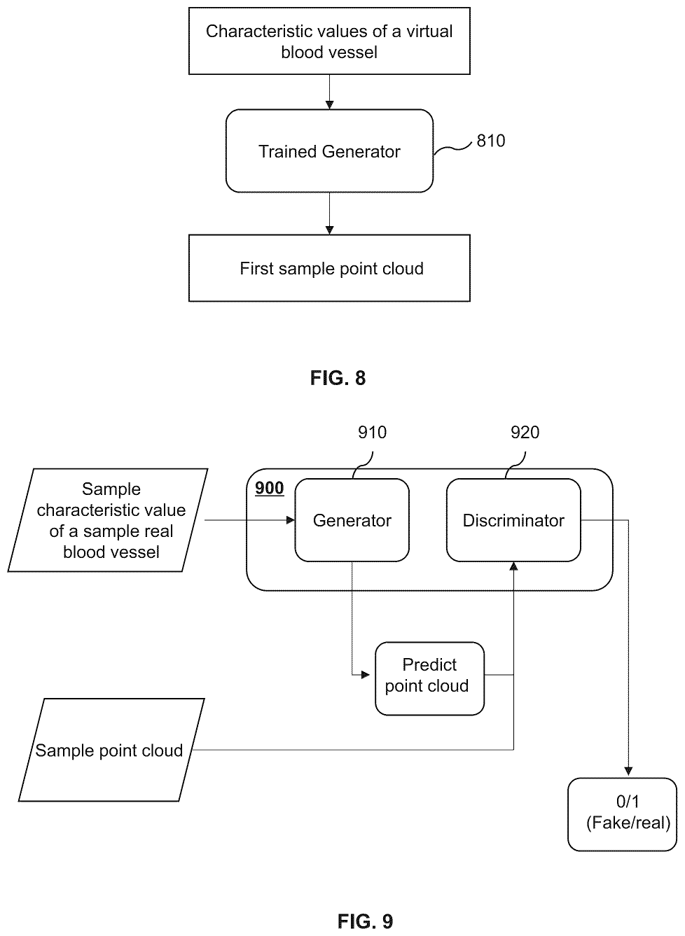

FIG. 8 is a schematic diagram illustrating an exemplary process for determining a first sample point cloud according to some embodiments of the present disclosure. As shown in FIG. 8. The processing device 120B may determine the first sample point cloud by inputting the one or more characteristic values of the virtual blood vessel into a trained generator 810. Merely by way of example, characteristic values of a virtual blood vessel, for example, [10; 5.5; 1.1; 1; 30] in which 10 represents the length of the virtual blood vessel, 5.5 represents the diameter of the virtual blood vessel, 1.1 represents the wall thickness of the virtual blood vessel, 1 represents that there is a stenosis in the virtual blood vessel, and 30 represents the ratio of the stenosis to the whole virtual blood vessel, may be input into the trained generator 810, the trained generator 810 may output a first sample point cloud of the virtual blood vessel. The first sample point cloud may include values of reference features of each blood vessel point of the virtual blood vessel, such as the diameter of the virtual blood vessel at the blood vessel point, whether there is a stenosis in the virtual blood vessel at the blood vessel point, a blood pressure of the virtual blood vessel at the blood vessel point, and a blood flow velocity of the virtual blood vessel at the blood vessel point. In some embodiments, the trained generator 810 may directly output the first sample point cloud including values of the reference features of each sample blood vessel point in the virtual blood vessel. Alternatively, the trained generator 810 may output an image representing the virtual blood vessel, and the processing device 120B may determine the first sample point cloud based on the image.

In some embodiments, the processing device 120B may obtain the trained generator by training, based on a plurality of second training samples, a generative adversarial network (GAN) including a generator and a discriminator. Each of the plurality of second training samples may include a sample characteristic value of a sample real blood vessel and a sample point cloud representing the sample real blood vessel. Merely by way of example, the sample characteristic value of the sample real blood vessel may include at least one of a length, a diameter, a diameter distribution, a start position, an end position, a curvature distribution, or lesion data of the sample real blood vessel, a function representing the diameter distribution, or a function representing curvature distribution. In some embodiments, in order to enable the trained generator to better learn characteristics of blood vessels with lesions, at least a portion of the plurality of second training samples may include the lesion data. The sample point cloud of the sample real blood vessel may be used as a training label, which may be determined in a similar manner as how the point cloud is determined as described in connection with operation 320 and confirmed or modified by a user.

FIG. 9 is a schematic diagram illustrating an exemplary process for determining a trained generator according to some embodiments of the present disclosure. As shown in FIG. 9, a GAN 900 may include a generator 910 and a discriminator 920. The sample characteristic value of a sample real blood vessel of a second training sample may be input into the generator 910, and the generator 910 may output a predicted point cloud representing the sample real blood vessel based on the input. The output of the generator 910 and the sample point cloud representing the sample real blood vessel may be input into the discriminator 920, and the discriminator 920 may output a value representing fake or real (e.g., 0 or 1, 0 representing fake, and 1 representing real). Specifically, the discriminator 920 may determine whether the predicted point cloud is true (i.e., the training label). If the predicted point cloud is true, the discriminator 920 may output 1; if the predicted point cloud is not true, the discriminator 920 may output 0. According to the output of the generator 920 and the output of the discriminator 920, the processing device 120B may optimize model parameters of the generator 910, until the generator 920 generates a predicted point cloud that can cheat the discriminator 920 and the discriminator 920 can distinguish false data from true data. In the embodiments, the training of the generator 910 is guided by the discriminator 920, so that the first sample point cloud generated using the trained generator is closer to a point cloud of a real blood vessel.

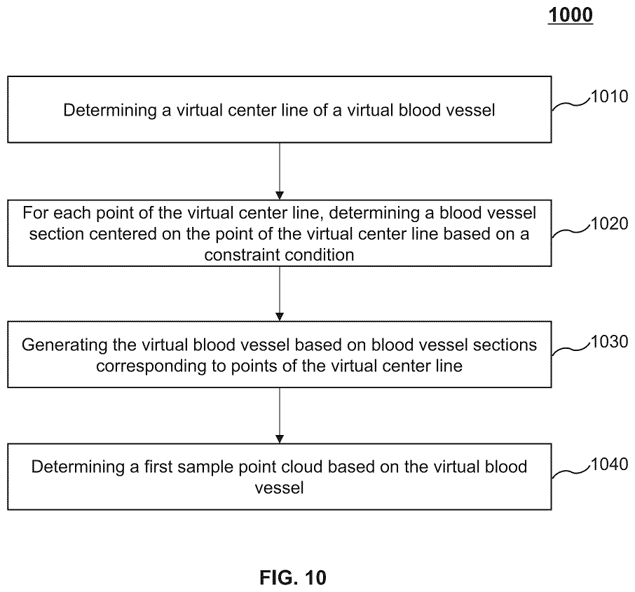

In some embodiments, the processing device 120B may determine a virtual center line of the virtual blood vessel and set a pipe diameter distribution for the virtual center line based on a type of the virtual blood vessel. The processing device 120B may generate the virtual blood vessel based on the virtual center line and the pipe diameter distribution, and determine the first sample point cloud based on the virtual blood vessel. More descriptions regarding the determination of the first sample point cloud may be found elsewhere in the present disclosure (e.g., FIG. 10 and the description thereof).

In 720, the processing device 120B (e.g., the training module 250) may generate the determination model by training a preliminary deep learning model (also referred to as a preliminary model for brevity) based on the plurality of training samples.

In some embodiments, the preliminary model may include one or more model parameters having one or more initial values before model training. The training of the preliminary model may include one or more iterations. For illustration purposes, the following descriptions are described with reference to a current iteration. When obtaining the determination model, in the current iteration, the processing device 120B may input the sample point cloud (e.g., the first sample point cloud or the second sample point cloud) representing the sample blood vessel points of the sample blood vessel of a training sample into the preliminary model (or an intermediate model obtained in a prior iteration (e.g., the immediately prior iteration)) in the current iteration to obtain predicted values of the one or more target features of the sample blood vessel points. The processing device 120B may determine a value of a loss function based on the predicted values and the ground truth values of the one or more target features of the sample blood vessel points. The loss function may be used to measure a difference between the predicted values and the ground truth values.

Further, the processing device 120B may determine whether a termination condition is satisfied in the current iteration based on the value of the loss function. Exemplary termination conditions may include that the value of the loss function obtained in the current iteration is less than a predetermined threshold, that a certain count of iterations is performed, that the loss function converges such that the differences of the values of the loss function obtained in consecutive iterations are within a threshold, or the like, or any combination thereof. In response to a determination that the termination condition is satisfied in the current iteration, the processing device 120B may designate the preliminary model in the current iteration as a trained model (e.g., the determination model). Further, the processing device 120B may store the trained model in a storage device (e.g., the storage device 130) of the medical system 100 and/or output the trained model for further use (e.g., in process 300). If the termination condition is not satisfied in the current iteration, the processing device 120B may update the preliminary model in the current iteration and proceed to a next iteration until the termination condition is satisfied.

In some embodiments, the determination model may have the structure shown in FIG. 11, and the training method of such determination model may be found in descriptions regarding FIG. 11.

FIG. 10 is a schematic diagram illustrating an exemplary process for determining a first sample point cloud according to some embodiments of the present disclosure. In some embodiments, the process 1000 may be performed to achieve at least part of operation 710 as described in connection with FIG. 7.

In 1010, the processing device 120B (e.g., the obtaining module 240) may determine a virtual center line of a virtual blood vessel.

The virtual center line of the virtual blood vessel may include points at the center line of the virtual blood vessel. The virtual center line of the virtual blood vessel may be straight or curved. In some embodiments, the processing device 120B may randomly obtain at least two points or obtain at least two points specified by a user (e.g., a doctor, an expert, etc.), and then determine the virtual center line of the virtual blood vessel by interpolation. In some embodiments, the virtual center line may be determined based on a center line of a true blood vessel.

In 1020, for each point of the virtual center line, the processing device 120B (e.g., the obtaining module 240) may determine a blood vessel section centered on the point of the virtual center line based on a constraint condition.

The constraint condition may include a diameter range, a wall thickness range, and/or a shape of the blood vessel section. The shape of the blood vessel section may include a regular shape such as a circle, an ellipse, or a crescent, or any irregular shape. The diameter range and/or the wall thickness range may be related to the type of the virtual blood vessel. Different types of virtual blood vessels may have different diameter ranges and/or wall thickness ranges. For example, the diameter range of the coronary artery is about two millimeters, for example, 1.8-2.2 millimeters, and the wall thickness range of the coronary artery is 0.1-0.9 microns.

In some embodiments, blood vessel sections corresponding to at least a portion of points of the virtual center line may be parallel to each other. In some embodiments, a blood vessel section corresponding to a point of the virtual center line may be perpendicular to a tangent line of the virtual center line at the point.

In 1030, the processing device 120B (e.g., the obtaining module 240) may generate the virtual blood vessel based on blood vessel sections corresponding to points of the virtual center line.

In some embodiments, the processing device 120B may generate the virtual blood vessel by superimposing blood vessel sections corresponding to all points of the virtual center line along the virtual center line. In some embodiments, before generating the virtual blood vessel, the processing device 120B may randomly add lesion data to at least a portion of the blood vessel sections. As described in connection with operation 710, the lesion data may include information related to a stenosis in the virtual blood vessel, for example, whether there is a stenosis in the virtual blood vessel, a ratio of the stenosis to the whole virtual blood vessel, a length of the stenosis, a position of the stenosis, a degree of the stenosis, etc. For example, the processing device 120B may adjust the portion of the blood vessel sections (e.g., the diameter range, the wall thickness range, and/or the shape of the blood vessel section) based on the lesion data. Merely by way of example, the processing device 120B may adjust blood vessel sections based on the length of the stenosis, the position of the stenosis, and the degree of the stenosis. In some embodiments, different generated virtual blood vessels may correspond to different types of lesion data, for example, different ratios of the stenosis to the whole virtual blood vessel, different lengths of the stenosis, different positions of the stenosis, different degrees of the stenosis, etc.

In 1040, the processing device 120B (e.g., the obtaining module 240) may determine the first sample point cloud based on the virtual blood vessel.

For example, for each sample blood vessel point of the virtual blood vessel, the processing device 120B may determine values of one or more reference features of the first sample blood vessel point.

By the above embodiments, a virtual blood vessel with local defects (e.g., the stenosis) whose features are close to the real blood vessel may be generated, so that the first sample point cloud may be determined without obtaining a blood vessel image, and the values of the one or more reference features of the first sample blood vessel point is evenly distributed. In addition, the above methods can generate virtual blood vessels with different lesions to provide more training samples for training the determination model, thereby improving the reliability of the generated determination model.

FIG. 11 is a schematic diagram illustrating an exemplary determination model according to some embodiments of the present disclosure.

As shown in FIG. 11, a determination model 1100 may include a point encoder 1110 and a point decoder 1120. In some embodiments, the point encoder 1110 may include a plurality of first convolution layers connected to each other and a plurality of first MLP layers connected to at least one of the plurality of first convolution layers. The point encoder 1110 may be configured to determine encoded first features of the plurality of blood vessel points and encoded second features of a plurality of blood vessel slices based on first features of the plurality of blood vessel points and second features of the plurality of blood vessel slices. A blood vessel where the plurality of blood vessel points are located may include a plurality of blood vessel slices distributed along an extension line of the blood vessel, and each of the plurality of blood vessel slices may include a portion of the plurality of blood vessel points. Optionally, each blood vessel slice may be perpendicular to the center line of the blood vessel. FIG. 12 is a schematic diagram illustrating exemplary blood vessel slices according to some embodiments of the present disclosure. As shown in FIG. 12, a blood vessel 1200 may include a plurality of blood vessel slices 1210 distributed along the extension line of the blood vessel 1200, and each of the plurality of blood vessel slices 1210 may include multiple blood vessel points. The first features may include the values of the reference features of the plurality of blood vessel points. The second features of each blood vessel slice may include the values of the reference features of blood vessel points in the blood vessel slice.

Specifically, as shown in FIG. 11, a point cloud 1140 includes first data points (i.e., the first features) representing the plurality of blood vessel points and second data points (i.e., the second features) representing blood vessel points of each blood vessel slice. The point cloud 1140 may be input into the point encoder 1110. The point encoder 1110 may output the encoded first features of the plurality of blood vessel points by performing feature extraction and transformation on the first features of the plurality of blood vessel points. Further, the point encoder 1110 may output the encoded second features of the plurality of blood vessel slices by performing chunk-pooling on the second features of the plurality of blood vessel slices. Specifically, the point encoder 1110 may determine a maximum value of the second features of each blood vessel slice by performing the chunk-pooling, and designate the maximum value as the encoded second features of the blood vessel slice.

The point decoder 1120 may be configured to determine, for each of the plurality of blood vessel points, the values of the one or more target features of the blood vessel point based on a combination of the encoded first features and the encoded second features. Specifically, the combination of the encoded first features and the encoded second features output by the point encoder 1110 may be input into the point decoder 1120, and the point decoder 1120 may output a plurality of data points 1150 corresponding to the blood vessel points. Each data point 1150 may include values of the one or more target features of the corresponding blood vessel point. In some embodiments, the point decoder 1120 may include a plurality of second convolution layers connected to each other and a plurality of second MLP layers connected to at least one of the plurality of second convolution layers. It should be noted that a first convolution layer may be the same as or different from a second convolution layer, a first MLP layer may be the same as or different from a second MLP layer.

In some embodiments, as shown in FIG. 11, the determination model 1100 may include a sequence encoder 1130. The sequence encoder 1130 may be configured to generate central features 1160 relating to central points of the plurality of blood vessel slices based on the second features and the encoded second features. Merely by way of example, the sequence encoder 1130 may include a recurrent neural network (RNN), a long short-term memory (LSTM), a gate recurrent unit (GRU), etc. Specifically, the sequence encoder 1130 may generate the central features 1160 by processing the second features and the encoded second features to determine features of the central points of the plurality of blood vessel slices and associating the features of the central points based on a blood flow direction of the blood vessel where the plurality of blood vessel points are located. Normally, blood flows direction is from a location with a high blood pressure to a location with a low blood pressure, and blood will sequentially pass through the central points of the plurality of blood vessel slices in a certain order. Therefore, the features of the central points (especially central points close to each other) are associated with each other. The sequence encoder 1130 may mine the association between the features of the central points in determining the central features 1160 relating to the central points, thereby improving the accuracy of the subsequently determined values of the one or more target features of each blood vessel point.

In the embodiments, the point decoder 1120 may be configured to determine the values of the one or more target features of each of the plurality of blood vessel points based on the combination of the encoded first features and the encoded second features and an up-sampling result of the central features 1160. Specifically, the combination of the encoded first features and the encoded second features output by the point encoder 1110 and the up-sampling result of the central features 1160 output by the sequence encoder 1130 may be input into the point decoder 1120, and the point decoder 1120 may output the plurality of data points 1150.

In some embodiments, in addition to the sample point cloud representing the sample blood vessel points of a sample blood vessel and the ground truth values of the one or more target features of the sample blood vessel points (e.g., as described in connection with FIG. 7), each of the plurality of training samples used to train the determination model may further include ground truth values of the one or more target features of central points of the sample blood vessel. During the training of the determination model 1100, a preliminary sequence encoder in a preliminary model (e.g., a preliminary deep learning model as described in connection with FIG. 7) is configured to determine predicted values of the one or more target features of the central points of the sample blood vessel in each iteration.

In some embodiments, a loss function used for training the determination model 1100 may include a point loss and optionally a sequence loss. The point loss may be related to the ground truth values of the one or more target features of the sample blood vessel points of the sample blood vessel. The point loss may be used to measure a difference between the ground truth values and predicted values of the one or more target features of the sample blood vessel points that are output by the preliminary model in each iteration. The sequence loss may be related to the ground truth values of the one or more target features of the central points of the sample blood vessel in the training sample. The sequence loss may be used to measure a difference between the ground truth values and the predicted values of the one or more target features of the central points of the sample blood vessel that are output by the preliminary sequence encode in the preliminary model in the each iteration.

By using the point loss and the sequence loss, the determination model 1100 can learn an optimized mechanisms for determining target feature(s) by mining not only associations between sample blood vessel points on the wall of a sample blood vessel, but also associations between central points of blood vessel slices of the sample blood vessel. Therefore, the determination model 1100 may have improved accuracy in determining values of the one or more target features of each blood vessel point and the values of the one or more target features of the central points of a blood vessel in an application.

The operations of the illustrated processes 300, 700, and 1000 presented above are intended to be illustrative. In some embodiments, a process may be accomplished with one or more additional operations not described, and/or without one or more of the operations discussed. Additionally, the order in which the operations of a process described above is not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” may mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C #, VB. NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (Saas).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate +1%, +5%, +10%, or +20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A method implemented on a computing device including at least one processor and at least one storage device, the method comprising:
obtaining a blood vessel image of a target subject;
generating, based on the blood vessel image, a point cloud including a plurality of data points representing a plurality of blood vessel points of the target subject, each of the plurality of data points including values of one or more reference features of the corresponding blood vessel point; and
for each of the plurality of blood vessel points, determining values of one or more target features of the blood vessel point based on the point cloud using a determination model, wherein the determination model is a trained deep learning model.

2. The method of claim 1, wherein the determination model is obtained by:
obtaining a plurality of training samples, wherein each of the plurality of training samples includes a sample point cloud representing sample blood vessel points of a sample blood vessel and ground truth values of the one or more target features of the sample blood vessel points, the sample blood vessel corresponding to at least one of the plurality of training samples is a virtual blood vessel; and
generating the determination model by training a preliminary deep learning model based on the plurality of training samples.

3. The method of claim 2, wherein the sample point cloud of a virtual blood vessel is determined using a trained generator based on one or more characteristic values of the virtual blood vessel.

4. The method of claim 3, wherein the trained generator is obtained by:
training, based on a plurality of second training samples, a generative adversarial network (GAN) including a generator and a discriminator, each of the plurality of second training samples including a sample characteristic value of a sample real blood vessel and a sample point cloud representing the sample real blood vessel.

5. The method of claim 2, wherein the sample point cloud of a virtual blood vessel is determined by:
determining a virtual center line of the virtual blood vessel;
for each point of the virtual center line, determining a blood vessel section centered on the point of the virtual center line based on a constraint condition;
generating the virtual blood vessel based on blood vessel sections corresponding to points of the virtual center line; and
determining the sample point cloud based on the virtual blood vessel.

6. The method of claim 1, wherein the determination model includes:
a PointNet configured to determine local features and global features of the plurality of blood vessel points based on the values of the reference features of the plurality of blood vessel points;
a recurrent neural network (RNN) configured to generate an output by processing the local features of the plurality of blood vessel points; and
a determination network configured to generate, for each of the plurality of blood vessel points, the values of the one or more target features of the blood vessel point based on the local features, the global features, and the output of the RNN.

7. The method of claim 1, wherein the determination model includes:
a point encoder configured to determine encoded first features of the plurality of blood vessel points and encoded second features of a plurality of blood vessel slices based on first features of the plurality of blood vessel points and second features of the plurality of blood vessel slices, the first features including the values of the reference features of the plurality of blood vessel points, the second features of each blood vessel slice including the values of the reference features of blood vessel points in the blood vessel slice; and
a point decoder configured to determine, for each of the plurality of blood vessel points, the values of the one or more target features of the blood vessel point based on a combination of the encoded first features and the encoded second features.

8. The method of claim 7, wherein:
the determination model further includes a sequence encoder configured to generate central features relating to central points of the plurality of blood vessel slices based on the second features and the encoded second features, and the point decoder is further configured to determine the values of the one or more target features of each of the plurality of blood vessel points based on the combination of the encoded first features and the encoded second features, and an up-sampling result of the central features.

9. The method of claim 8, wherein
each of a plurality of training samples configured to train the determination model includes ground truth values of the one or more target features of central points of a sample blood vessel, and
during the training of the determination model, a preliminary sequence encoder in a preliminary deep learning model is configured to determine predicted values of the one or more target features of the central points of the sample blood vessel in each iteration.

10. The method of claim 9, wherein a loss function used for training the determination model includes:
a point loss related to ground truth values of the one or more target features of sample blood vessel points of the sample blood vessel, and
a sequence loss related to the ground truth values of the one or more target features of the central points of the sample blood vessel.

11. The method of claim 1, wherein for each of the plurality of blood vessel points, the determining the values of the one or more target features of the blood vessel point based on the point cloud using the determination model includes:
for each of one or more reference features of the blood vessel point, determining a weight of the reference feature based on a position of the blood vessel point in a blood vessel corresponding to the blood vessel point; and
determining the values of the one or more target features of the blood vessel point based on values and weights of reference features of the plurality of blood vessel points using the determination model.

12. The method of claim 1, wherein for each of the plurality of blood vessel points, the determining the values of the one or more target features of the blood vessel point based on the point cloud using the determination model includes:
dividing one or more reference features of the blood vessel point into a plurality of reference feature sets;
for each of the plurality of reference feature sets,
determining a weight of the reference feature set based on a position, in a blood vessel corresponding to the blood vessel point, of the blood vessel point;
determining a candidate value set of the one or more target features of the blood vessel point based on values of reference features in the reference feature set using the determination model; and
determining the values of the one or more target features of the blood vessel point based on candidate value sets and weights corresponding to the plurality of reference feature sets.

13. A system, comprising:
at least one storage device including a set of instructions; and
at least one processor in communication with the at least one storage device, wherein when executing the set of instructions, the at least one processor causes the system to perform operations including:
obtaining a blood vessel image of a target subject;
generating, based on the blood vessel image, a point cloud including a plurality of data points representing a plurality of blood vessel points of the target subject, each of the plurality of data points including values of one or more reference features of the corresponding blood vessel point; and
for each of the plurality of blood vessel points, determining values of one or more target features of the blood vessel point based on the point cloud using a determination model, wherein the determination model is a trained deep learning model.

14. The system of claim 13, wherein the determination model is obtained by:
obtaining a plurality of training samples, wherein each of the plurality of training samples includes a sample point cloud representing sample blood vessel points of a sample blood vessel and ground truth values of the one or more target features of the sample blood vessel points, the sample blood vessel corresponding to at least one of the plurality of training samples is a virtual blood vessel; and
generating the determination model by training a preliminary deep learning model based on the plurality of training samples.

15. The system of claim 14, wherein the sample point cloud of a virtual blood vessel is determined using a trained generator based on one or more characteristic values of the virtual blood vessel.

16. The system of claim 15, wherein the trained generator is obtained by:
training, based on a plurality of second training samples, a generative adversarial network (GAN) including a generator and a discriminator, each of the plurality of second training samples including a sample characteristic value of a sample real blood vessel and a sample point cloud representing the sample real blood vessel.

17. The system of claim 14, wherein the sample point cloud of a virtual blood vessel is determined by:
determining a virtual center line of the virtual blood vessel;
for each point of the virtual center line, determining a blood vessel section centered on the point of the virtual center line based on a constraint condition;
generating the virtual blood vessel based on blood vessel sections corresponding to points of the virtual center line; and
determining the sample point cloud based on the virtual blood vessel.

18. The system of claim 13, wherein the determination model includes:
a PointNet configured to determine local features and global features of the plurality of blood vessel points based on the values of the reference features of the plurality of blood vessel points;
a recurrent neural network (RNN) configured to generate an output by processing the local features of the plurality of blood vessel points; and
a determination network configured to generate, for each of the plurality of blood vessel points, the values of the one or more target features of the blood vessel point based on the local features, the global features, and the output of the RNN.

19. The system of claim 13, wherein the determination model includes:
a point encoder configured to determine encoded first features of the plurality of blood vessel points and encoded second features of a plurality of blood vessel slices based on first features of the plurality of blood vessel points and second features of the plurality of blood vessel slices, the first features including the values of the reference features of the plurality of blood vessel points, the second features of each blood vessel slice including the values of the reference features of blood vessel points in the blood vessel slice; and
a point decoder configured to determine, for each of the plurality of blood vessel points, the values of the one or more target features of the blood vessel point based on a combination of the encoded first features and the encoded second features.

20. A non-transitory computer readable medium, comprising executable instructions that, when executed by at least one processor, direct the at least one processor to perform a method, the method comprising:
obtaining a blood vessel image of a target subject;
generating, based on the blood vessel image, a point cloud including a plurality of data points representing a plurality of blood vessel points of the target subject, each of the plurality of data points including values of one or more reference features of the corresponding blood vessel point; and
for each of the plurality of blood vessel points, determining values of one or more target features of the blood vessel point based on the point cloud using a determination model, wherein the determination model is a trained deep learning model.
